(19) United States
(12) Patent Application Publication
(10) Pub. No.: US 2013/0245064 A1
(43) Pub. Date: Sep. 19, 2013

(54) NOVEL NEUROTRYPSIN INHIBITORS

(71) Applicants: Shaheen Ahmed, Zurich (CH); Stefan Hettwer, Rupperswil (CH); Jan Willem Vrijbloed, Mohlin (CH); Carlo Farina, Valsolda (IT); Piero Paravidino, Sedriano (IT)

(72) Inventors: Shaheen Ahmed, Zurich (CH); Stefan Hettwer, Rupperswil (CH); Jan Willem Vrijbloed, Mohlin (CH); Carlo Farina, Valsolda (IT); Piero Paravidino, Sedriano (IT)

(21) Appl. No.: 13/882,345

(22) PCT Filed: Oct. 31, 2011
(86) PCT No.: PCT/EP2011/069075

§ 371 Date: Jun. 5, 2013

(30) Foreign Application Priority Data
Nov. 1, 2010 (EP) 10189541.5

(51) Int. Cl. C07C 237/52; C07D 209/42; C07D 401/12; C07D 333/38; C07D 215/48; C07D 235/18; C07D 333/70

(52) U.S. Cl. CPC C07C 237/52; C07D 235/18; C07D 209/42; C07D 333/70; C07D 333/38; C07D 215/48; C07D 401/12
USPC 514/311; 548/310.7; 548/492; 564/158; 549/57; 549/72; 546/169; 546/199; 514/394; 514/419; 514/616; 514/443; 514/448; 514/322

(57) ABSTRACT

The invention relates to novel acylamino-hydroxy-benzamides of formula (I), wherein R¹ is phenyl substituted by phenyl, phenoxy, phenylamino or heteroaryl, all optionally further substituted; bicyclic aryl, monocyclic heteroaryl substituted by optionally substituted phenyl, or bicyclic heteroaryl, R² is hydrogen or methyl, and R³ and R⁴ have the meanings indicated in the description. These compounds are useful for the treatment and/or prophylaxis of skeletal muscle atrophy, schizophrenia and Alzheimer's disease, and as cognitive enhancers.

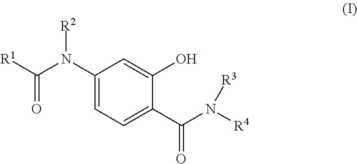

### FIELD OF THE INVENTION

The invention relates to novel acylamino-hydroxy-benzamides, and the use of such compounds for the treatment and/or prophylaxis of skeletal muscle atrophy, schizophrenia and Alzheimer's disease, and as cognitive enhancers.

### BACKGROUND OF THE INVENTION

The enzyme neurotrypsin (WO 98/49322) belongs to the chymotrypsin family, whose members are almost entirely confined to animals. The amino acid sequence of neurotrypsin defines a mosaic protein of 875 amino acids consisting of a Kringle domain, followed by four scavenger receptor cysteine-rich repeats (three in the mouse), and the serine protease domain. Neurotrypsin contains, like thrombin, tPA, trypsin and some other enzymes, an aspartate residue in the bottom of its S1 pocket, therefore showing specificity for basic amino acids at this binding site. The structural similarity of neurotrypsin to the proteases of the blood coagulation cascade and the fibrinolytic system, such as factor X, factor IX, thrombin, tissue plasminogen activator, and plasmin suggests that it may be an element of a protease-driven extracellular signaling mechanism in the nervous system.

As was shown in WO 2006/103261, neurotrypsin is located at the presynaptic nerve terminal of synapses of the central nervous system (CNS) and at the neuromuscular junction (NMJ). The synapse is the connection between nerve cells (neurons) where messages are communicated in the form of chemical substances, termed neurotransmitters. The synapse is composed of a presynaptic terminal formed by the signal-emitting cell and the postsynaptic specialization of the signal-receiving cell. Neurotransmitters released from the presynaptic terminal cross the synaptic cleft and bind to the neurotransmitter receptors in the postsynaptic specialization. Upon binding of the neurotransmitter the receptor induces the generation of an electrical pulse in the postsynaptic cell. Signal transmission between two neurons is the basis of neuronal function. Brain functions are the result of the specific assembly of an enormous number of neurons to information-processing networks.

The majority of synapses is found in the central nervous system (CNS, brain and spine), where every synapse connects two neurons. By such bilateral point-to-point connections, every neuron may connect to thousands of other neurons. However, synapses also connect a neuron to a gland or a muscle cell. The neuromuscular junction (NMJ, muscle end-plate) is the synapse that connects a nerve cell with a striated muscle cell. Synapses located outside of the brain, the brain stem and the spinal cord are termed peripheral nervous system (PNS) synapses. CNS synapses and PNS synapses exhibit many structural and functional commonalities and share many of their molecular components (synaptic molecules). Therefore, synaptic target molecules may be useful for targeting synaptic functions of both the CNS and the PNS.

Skeletal muscle atrophy (sarcopenia), defined as the loss of muscle mass and strength, plays a major role in the pathogenesis of frailty and functional impairment that occurs with old age. It plays a major role in the loss of muscular strength, decreased metabolic rate, gradual reduction of bone density and decreased aerobic capacity (Doherty, T. J., J. Appl. Physiol. 95: 1717-1727, 2003). The loss of muscle mass manifests as a decrease in the cross-sectional area of the muscle with age, which has been determined to result from a combined effect of a reduction in both the number of muscle fibers and the thickness of the individual remaining fibers.

Over the past years, considerable progress has been made in the identification and characterization of factors contributing to the degradation of muscle mass. Important genes associated with these processes encode ubiquitin protein ligases that were found increased in atrophic muscle. Among the factors that have a hypertrophic activity and, as such, block atrophy, insulin-like growth factor 1 (IGF-1) has been found to play an essential role. This and several other regulatory pathways controlling skeletal muscle mass have been investigated intensively (for a review see: Glass, D. J., Nature Cell Biol. 5: 87-90, 2003). In spite of important progress in both the characterization of the molecular mechanisms that control muscle degradation leading to atrophy and the hypertrophic effects of insulin-like growth factor, and in spite of the fact that several companies work on the development of drugs capable of stimulating the increase of muscle mass, no drugs have been approved up to now.

A morphological hallmark of the skeletal muscle atrophy found at old age (sarcopenia) is a considerable reduction of the number of muscle fibers. Ample evidence from numerous independent studies supports that neural input to a fraction of the muscle fibers is disrupted with age, resulting in subsequent atrophy and eventually the disappearance of the denervated fibers (Kamal, H. K., Nutrition Reviews 61: 157-167, 2003). Another characteristic feature of the skeletal muscle atrophy found at old age is a coincidence of the muscular atrophy with a considerable reduction of the number of motoneurons (VVelle, S., Can. J. Appl. Physiol. 27: 19-41, 2002) and a marked structural alteration of the neuromuscular junction (Tapia, J. C. et al., Abstract Viewer/Itinerary Planner, Washington D.C.: Society for Neuroscience). These characteristics indicate that a significant age-related deterioration of the structure and the function of the neuromuscular junction is a major contributing factor to a process that ultimately results in a structural and functional denervation. Denervated muscle fibers that do not receive compensatory reinnervation within weeks become progressively atrophic and eventually disappear.

Schizophrenia is a chronic, severe, and disabling brain disease. Approximately 1% of the world population develops schizophrenia during their lifetime. Individuals who develop schizophrenia experience severe suffering. Approximately 10% commit suicide. Although schizophrenia affects men and women with equal frequency, the disorder often appears earlier in men, usually in the late teens or early twenties, than in women, who are generally affected in the twenties to early thirties. People with schizophrenia often suffer terrifying symptoms such as hearing internal voices not heard by others, or believing that other people are reading their minds, controlling their thoughts, or plotting to harm them. These symptoms may leave them fearful and withdrawn. Their speech and behavior can be so disorganized that they may be incomprehensible or frightening to others. The currently available treatments of schizophrenia reduce suffering considerably, but approximately ⅔ of the people affected by schizophrenia require public assistance within a few years after onset. The majority of them are unable to return to work or school and have relatively little or no social interactions, and most people with schizophrenia continue to suffer some symptoms throughout their lives. It has been estimated that no more than one in five individuals recovers completely. Therefore schizophrenia is one of the most important public health problems world-wide, and the costs to society are counted in billions of dollars.

Alzheimer's disease (AD) is the most common form of dementia whose most common symptoms include inability to acquire new memories, difficulty in recalling recently observed facts, confusion, irritability and aggression, mood swings, language breakdown and long-term memory loss. The cause and progression of Alzheimer's disease are not well understood. Research indicates that the disease is associated with plaques and tangles in the brain. Recently, it has been shown that loss of lean mass is accelerated in AD and is associated with brain atrophy and cognitive performance, perhaps as a direct or indirect consequence of AD pathophysiology or through shared mechanisms common to both AD and sarcopenia. (Burns et al., Arch Neurol. 67: 428-33, 2010). Therefore, inhibitors of neurotrypsin could also be effective in treating Alzheimer's disease or in reducing its symptoms.

The currently most consistent neuropathological finding in brains of schizophrenic patients is a reduction of the number of synapses in the gray matter of the central nervous system, which is reflected by a decrease in the volume of the neuropil (the synaptic area). No evidence for neuronal degeneration is observed. Typically, the number of neurons counted per area of tissue is rather increased, an observation explained by a selective decrease in the number of synapses in the neuropil area between the neurons while the number of neuronal cell somas remained constant. The phenomenon has been reported over the past two decades by several independent studies on post mortem material and has been found most extensive in the prefrontal cortex. The literature documenting this observation has been carefully reviewed by Selemon, L. D. and Goldman-Rakic, P. S. (Psychiatry 45: 17-25, 1999). McGlashan, T. H. and Hoffman, R. E. (Arch. Gen. Psychiatry 57: 637-648, 2000) summarized the essential morphological, developmental, electrophysiological, and metabolic observations in schizophrenia in the light of the “excessive synaptic pruning” hypothesis and came to the conclusion that “excessive synaptic pruning” or “developmentally reduced synaptic connectivity” is an increasingly attractive pathophysiological model of schizophrenia. Based on this model, schizophrenia arises from critically reduced synaptic connectedness as a result of developmental disturbances of synaptogenesis during gestation and early childhood and/or excessive synaptic pruning during adolescence. The model accounts for the phenomenology of the disorder, the symptomatic states, the onset, neurodevelopmental deficits, window of deterioration, sex differences in clinical presentation, course determined by age of onset, and preservation of the schizophrenic genotype in the population despite diminished phenotypic fecundity.

Cognitive enhancers are drugs aimed at preventing, improving, or treating cognitive deficits at both the clinical and subclinical level. Such drugs are beneficial for the treatment of memory difficulties of elderly people who have not progressed to Alzheimer's disease (mild cognitive impairment). However, such drugs are also beneficial for the improvement of cognitive functions in patients with the established diagnosis of Alzheimer's disease or other diseases associated with dementia or for the improvement of cognitive functions in posttraumatic cognitive dysfunction, as well as for the improvement of the age-related impairment of cognitive functions that are considered as a normal feature of the ageing process.

Mild cognitive impairment is a widely cited concept in clinical research on ageing-related cognitive disorders (Ritchie, K. and Touchon, J., The Lancet 355: 225-228, 2000). It refers generally to subclinical complaints of memory functioning in elderly people, which are judged to have a high probability of evolving towards Alzheimer's disease. The identification of people at potential risk for dementia with a view to early therapeutic intervention is important, because it may lessen distress for both patient and family, minimize the risk of accidents, prolong autonomy, and perhaps even ultimately prevent the onset of the process leading to dementia itself.

The impairment of cognitive functions without dementia is so common among elderly people that it is considered by many as an inevitable feature of the ageing process. Nonetheless, it has acquired clinical significance because of the difficulties patients may have with carrying out everyday activities. Although the range of impairments seen in populations without dementia is extremely broad, several clinical labels have been proposed to describe this tail-end of the normal cognitive range. One of the earliest was benign senescent forgetfulness. Its clinical features include an inability to recall minor detail, the forgetting of remote as opposed to recent events, and awareness of memory problems. The term ageing-associated cognitive decline refers to a wider range of cognitive functions (attention, memory, learning, thinking, language, and visuospatial function), and is diagnosed by reference to norms for elderly people. Prescription of cognitive enhancers may prolong the capacity of the affected individuals to carry out their daily activities and, thus, prolong their autonomy. Other disorders associated at least in part of the affected individuals with cognitive impairments that may eventually lead to dementia include Parkinson's disease, multiple sclerosis, stroke, and head trauma. The prescription of cognitive enhancer drugs may also improve cognitive functions in these patients.

### SUMMARY OF THE INVENTION

The invention relates to compounds of formula (I)

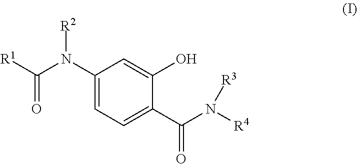

wherein
R¹ is phenyl substituted by phenyl, phenoxy, phenylamino or heteroaryl, all optionally further substituted; bicyclic aryl, monocyclic heteroaryl substituted by optionally substituted phenyl, or bicyclic heteroaryl;
R² is hydrogen or methyl;
R³ is alkyl, optionally substituted amino- or hydroxy-alkyl; aryl-lower alkyl, or aryl; and
R⁴ is hydrogen, lower alkyl, carboxy-, lower alkoxycarbonyl-, dimethylcarbamoyl-, hydroxy- or lower alkoxy-lower alkyl; or
R³ and R⁴ together with the nitrogen atom, to which they are bound, are optionally substituted pyrrolidino, optionally substituted piperidino, tetrahydro-quinolyl or -isoquinolyl, morpholino, or optionally substituted piperazino.

The invention further relates to compounds as defined hereinbefore for use as medicaments, in particular for use in the treatment and/or prophylaxis of diseases caused by deficiency of synapses, for example skeletal muscle atrophy, schizophrenia, Alzheimer's disease and cognitive disturbance, pharmaceutical preparations containing these compounds, and a method of treatment and/or prophylaxis of diseases caused by deficiency of synapses, for example skeletal muscle atrophy, schizophrenia, Alzheimer's disease and cognitive disturbance.

### DETAILED DESCRIPTION OF THE INVENTION

The invention is based on the fact that inhibition of neurotrypsin allows enhancing pro-synaptic (synapse-forming, synapse-differentiating, synapse-organizing, synapse-protecting, synapse-strengthening) activities. The neurotrypsin gene is expressed in many neurons of the central nervous system, including the motoneurons of the spinal cord, and the neurotrypsin protein is found in many CNS synapses, as well as at the neuromuscular junction. Neurotrypsin plays a substantial role in the development and/or the maintenance of a well balanced synaptic function. Too much neurotrypsin (overexpression) correlates with too few synaptic connections. Transgenic mice overexpressing neurotrypsin in CNS neurons show a reduced number of synapses in the cerebral cortex and the hippocampus, two brain structures that are highly important for cognitive functions, such as memory and learning. Likewise, transgenic mice overexpressing neurotrypsin in spinal motoneurons show a reduction of the neuromuscular junctions, the synapses that mediate the neural control of muscular activity (WO 2006/103261).

The pharmaceutical tuning of neurotrypsin activity provides an unprecedented access to the regulatory machinery of synaptic function. Inhibiting proteolytic activity of neurotrypsin shifts the synaptic balance towards strengthening the pro-synaptic activities at the expense of the anti-synaptic activities and thus towards increasing the number and/or the size and/or the strength of synapses.

The invention relates to compounds of formula (I)

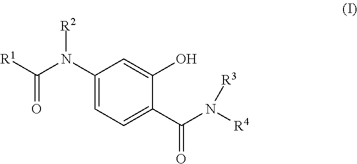

wherein
R¹ is phenyl substituted by phenyl, phenoxy, phenylamino or heteroaryl, all optionally further substituted; bicyclic aryl, monocyclic heteroaryl substituted by optionally substituted phenyl, or bicyclic heteroaryl;
R² is hydrogen or methyl;
R³ is alkyl, optionally substituted amino- or hydroxy-alkyl; aryl-lower alkyl, or aryl; and
R⁴ is hydrogen, lower alkyl, carboxy-, lower alkoxycarbonyl-, dimethylcarbamoyl-, hydroxy- or lower alkoxy-lower alkyl; or
R³ and R⁴ together with the nitrogen atom, to which they are bound, are optionally substituted pyrrolidino, optionally substituted piperidino, tetrahydro-quinolyl or -isoquinolyl, morpholino, or optionally substituted piperazino;
and pharmaceutically acceptable salts thereof.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts, and the like, this is taken to mean also a single compound, salt, or the like.

Double bonds in principle can have E- or Z-configuration. The compounds of this invention may therefore exist as isomeric mixtures or single isomers. If not specified both isomeric forms are intended.

Any asymmetric carbon atoms may be present in the (R)-, (S)- or (R,S)-configuration, preferably in the (R)- or (S)-configuration. The compounds may thus be present as mixtures of isomers or as pure isomers, preferably as enantiomer-pure diastereomers.

The invention relates also to possible tautomers of the compounds of formula (I).

Alkyl has from 1 to 12, preferably from 1 to 7 carbon atoms, and is linear or branched. Alkyl is preferably lower alkyl.

Lower alkyl has 1 to 7, preferably 1 to 4 carbon atoms and is butyl, such as n-butyl, sec-butyl, iso-butyl, tert-butyl, propyl, such as n-propyl or iso-propyl, ethyl or methyl. Preferably lower alkyl is methyl or ethyl. Lower alkyl may also be designated as C₁-C₇-alkyl, preferably C₁-C₄-alkyl.

Cycloalkyl has preferably 3 to 7 ring carbon atoms, and may be unsubstituted or substituted, e.g. by lower alkyl or lower alkoxy. Cycloalkyl is, for example, cyclohexyl, cyclopentyl, methylcyclopentyl, cycloheptyl or cyclopropyl.

Aryl stands for a mono- or bicyclic fused ring aromatic group with 5 to 10 carbon atoms, such as phenyl, 1-naphthyl or 2-naphthyl, or also a partially saturated bicyclic fused ring comprising a phenyl group, for example benzo-C₅- or —C₆-cycloalkyl or -cycloalkenyl, such as indanyl, indenyl, dihydro- or tetrahydronaphthyl. Preferably, aryl is phenyl or benzo-C₅— or —C₆-cycloalkyl, in particular phenyl.

Aryl is unsubstituted or substituted. Aryl may be substituted by up to four substituents independently selected from lower alkyl, halo-lower alkyl, cycloalkyl-lower alkyl, carboxy-lower alkyl, lower alkoxycarbonyl-lower alkyl; arylalkyl or heteroarylalkyl, wherein aryl or heteroaryl are unsubstituted or substituted by up to three substituents selected from lower alkyl, halo-lower alkyl, lower alkoxy, halogen, amino, cyano and nitro; hydroxy-lower alkyl, lower alkoxy-lower alkyl, aryloxy-lower alkyl, heteroaryloxy-lower alkyl, aryl-lower alkoxy-lower alkyl, heteroaryl-lower alkoxy-lower alkyl, lower alkoxy-lower alkoxy-lower alkyl; aminoalkyl wherein amino is unsubstituted or substituted by one or two substituents selected from lower alkyl, hydroxy-lower alkyl, alkoxy-lower alkyl, amino-lower alkyl, alkylcarbonyl, alkoxycarbonyl, amino-lower alkoxycarbonyl, lower alkoxy-lower alkoxy-carbonyl and carbamoyl (i.e. aminocarbonyl), or wherein the two substituents on nitrogen form together with the nitrogen heterocyclyl; optionally substituted alkenyl, optionally substituted alkinyl, cycloalkyl, aryl, heteroaryl, heterocyclyl, hydroxy, lower alkoxy, halo-lower alkoxy, lower alkoxy-lower alkoxy, cycloalkyl-lower alkoxy, aryloxy, aryl-lower alkoxy, aryloxy-lower alkoxy, heteroaryloxy, heteroaryl-lower alkoxy, heteroaryloxy-lower alkoxy, optionally substituted alkenyloxy, optionally substituted alkinyloxy, cycloalkyloxy, heterocyclyloxy, alkylmercapto, alkylsulfinyl, halo-lower alkylsulfinyl, alkylsulfonyl, arylsulfonyl, heteroarylsulfonyl, guanidinosulfonyl; sulfamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted phenyl, optionally substituted phenyl-lower alkyl, optionally substituted heteroaryl, optionally substituted heteroaryl-lower alkyl, alkylcarbonyl, alkoxycarbonyl or carbamoyl, and wherein alkyl or lower alkyl in each case may be substituted by halogen, lower alkoxy, aryl, heteroaryl or optionally substituted amino, or wherein the two substituents on nitrogen form together with the nitrogen heterocyclyl; lower alkylcarbonyl, halo-lower alkylcarbonyl, optionally substituted phenylcarbonyl, optionally substituted heteroarylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; cyano, halogen, and nitro; and wherein two substituents in ortho-position to each other can form a 5-, 6- or 7-membered carbocyclic or heterocyclic ring containing one, two or three oxygen atoms, one or two nitrogen atoms and/or one sulfur atom.

In particular, the substituents may be independently selected from lower alkyl, halo-lower alkyl, hydroxy-lower alkyl, lower alkoxy-lower alkyl, optionally substituted alkenyl, optionally substituted alkinyl, cyclohexyl, aryl, heteroaryl, heterocyclyl, hydroxy, lower alkoxy, halo-lower alkoxy, lower alkoxy-lower alkoxy, cycloalkyloxy, optionally substituted phenyloxy, optionally substituted phenyl-lower alkoxy; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted heteroaryl, alkylcarbonyl, alkoxycarbonyl or carbamoyl, or wherein the two substituents on nitrogen form together with the nitrogen heterocyclyl; lower alkylcarbonyl, halo-lower alkylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, hydroxy-lower alkyl, lower alkoxy-lower alkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; arylsulfonyl, heteroarylsulfonyl, guanidinosulfonyl, sulfamoyl, cyano, halogen, and nitro; and wherein two substituents in ortho-position to each other can form a 5- or 6-membered heterocyclic ring containing one or two oxygen atoms and/or one nitrogen atom.

In optionally substituted phenyl, phenoxy or phenylamino, substituents are preferably lower alkyl, halo-lower alkyl, lower alkoxy-lower alkyl, hydroxy, lower alkoxy, halo-lower alkoxy, lower alkoxy-lower alkoxy, methylenedioxy or ethylenedioxy, sulfamoyl, lower alkyl- or di-lower alkyl-sulfamoyl, guanidinosulfonyl, halo, carboxy, lower alkoxycarbonyl, cyano or nitro.

Heteroaryl represents an aromatic group containing at least one heteroatom selected from nitrogen, oxygen and sulfur, and is mono- or bicyclic. Monocyclic heteroaryl includes 5 or 6 membered heteroaryl groups containing 1, 2, 3 or 4 heteroatoms selected from nitrogen, sulfur and oxygen. Bicyclic heteroaryl includes 9 or 10 membered fused-ring heteroaryl groups. Examples of heteroaryl include pyrrolyl, thiophenyl (i.e. thienyl), furyl, pyrazolyl, imidazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, and benzo fused derivatives of such monocyclic heteroaryl groups, such as indolyl, benzimidazolyl, benzothiophenyl or benzothiazolyl, quinolinyl, isoquinolinyl, quinazolinyl, or purinyl. Preferably, heteroaryl is pyrrolyl, thiophenyl, pyrazolyl, imidazolyl, triazolyl, tetrazolyl, oxazolyl, isoxazolyl, thiazolyl, pyridyl, pyridazinyl, pyrimidinyl, or pyrazinyl, in particular thiophenyl, benzothiophenyl, pyrazolyl, imidazolyl, benzimidazolyl, triazolyl, tetrazolyl, isoxazolyl, thiazolyl, benzo-thiazolyl, pyridyl, or pyridazinyl.

Heteroaryl is unsubstituted or substituted. Heteroaryl may be substituted by up to three substituents independently selected from lower alkyl, halo-lower alkyl, cycloalkyl-lower alkyl, hydroxy-lower alkyl, lower alkoxy-lower alkyl, aryloxy-lower alkyl, heteroaryloxy-lower alkyl, lower alkoxy-lower alkoxy-lower alkyl; aminoalkyl, wherein amino is unsubstituted or substituted by one or two substituents selected from lower alkyl, hydroxy-lower alkyl, alkoxy-lower alkyl, amino-lower alkyl, alkylcarbonyl, alkoxycarbonyl, amino-lower alkoxycarbonyl, lower alkoxy-lower alkoxycarbonyl and carbamoyl; optionally substituted alkenyl, optionally substituted alkinyl, cycloalkyl; aryl, heteroaryl, arylalkyl or heteroarylalkyl, wherein aryl or heteroaryl are unsubstituted or substituted by up to three substituents selected from lower alkyl, halo-lower alkyl, lower alkoxy, halogen, amino, cyano and nitro; hydroxy, lower alkoxy, halo-lower alkoxy, lower alkoxy-lower alkoxy, cycloalkyloxy, cycloalkyl-lower alkoxy, aryloxy, aryl-lower alkoxy, heteroaryloxy, heteroaryl-lower alkoxy, alkenyloxy, alkinyloxy, alkylmercapto, alkylsulfinyl, halo-lower alkylsulfinyl, alkylsulfonyl, arylsulfonyl, heteroarylsulfonyl, sulfamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, optionally substituted phenyl, optionally substituted phenyl-lower alkyl, optionally substituted heteroaryl, optionally substituted heteroaryl-lower alkyl, alkylcarbonyl, alkoxycarbonyl or carbamoyl, and wherein alkyl or lower alkyl in each case may be substituted by halogen, lower alkoxy, aryl, heteroaryl or optionally substituted amino, or wherein the two substituents on nitrogen form together with the nitrogen heterocyclyl; lower alkylcarbonyl, halo-lower alkylcarbonyl, optionally substituted phenylcarbonyl, carboxy, lower alkoxycarbonyl, lower alkoxy-lower alkoxycarbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl, cycloalkyl, optionally substituted phenyl, optionally substituted phenyl-lower alkyl, optionally substituted heteroaryl and optionally substituted heteroaryl-lower alkyl, or wherein the two substituents on nitrogen form together with the nitrogen heterocyclyl; cyano, halogen, and nitro.

In particular, the substituents on heteroaryl may be independently selected from lower alkyl, halo-lower alkyl, cycloalkyl-lower alkyl, lower alkoxy-lower alkyl, lower alkoxy-lower alkoxy-lower alkyl, optionally substituted alkenyl, optionally substituted alkinyl, cycloalkyl, aryl, heteroaryl, hydroxy, lower alkoxy, cycloalkyloxy, alkenyloxy, alkinyloxy, alkyl-mercapto, alkylsulfinyl, halo-lower alkylsulfinyl, alkylsulfonyl, arylsulfonyl; amino optionally substituted by one or two substitutents selected from lower alkyl, cycloalkyl-lower alkyl, hydroxy-lower alkyl, lower alkoxy-lower alkyl, di-lower alkylamino-lower alkyl, cycloalkyl, alkylcarbonyl, alkoxycarbonyl or carbamoyl, and wherein alkyl or lower alkyl in each case may be substituted by lower alkoxy or optionally substituted amino, or wherein the two substituents on nitrogen form together with the nitrogen heterocyclyl; lower alkylcarbonyl, halo-lower alkylcarbonyl, carboxy, lower alkoxycarbonyl, lower alkoxy-lower alkoxy-carbonyl; carbamoyl wherein the nitrogen atom is unsubstituted or substituted by one or two substitutents selected from lower alkyl, cycloalkyl-lower alkyl, hydroxy-lower alkyl, lower alkoxy-lower alkyl or cycloalkyl, or wherein the two substituents on nitrogen form together with the nitrogen heterocyclyl; cyano, halogen, and nitro.

Preferred substituents in heteroaryl are lower alkyl, halo-lower alkyl, lower alkoxy-lower alkyl, hydroxy, lower alkoxy, halo-lower alkoxy, lower alkoxy-lower alkoxy, methylenedioxy, halo, carboxy, cyano, nitro, or optionally substituted phenyl.

Alkenyl contains one or more, e.g. two or three, double bonds, and is preferably lower alkenyl, such as 1- or 2-butenyl, 1-propenyl, allyl or vinyl.

Alkinyl is preferably lower alkinyl, such as propargyl or acetylenyl.

In optionally substituted alkenyl or alkinyl, substituents are preferably lower alkyl, lower alkoxy, halo, optionally substituted aryl or optionally substituted heteroaryl, and are connected with a saturated or unsaturated carbon atom of alkenyl or alkinyl.

Heterocyclyl designates preferably a saturated, partially saturated or unsaturated, mono- or bicyclic ring containing 4-10 atoms comprising one, two or three heteroatoms selected from nitrogen, oxygen and sulfur, which may, unless otherwise specified, be carbon or nitrogen linked, wherein a ring nitrogen atom may optionally be substituted by a group selected from lower alkyl, amino-lower alkyl, aryl, aryl-lower alkyl and acyl, and a ring carbon atom may be substituted by lower alkyl, amino-lower alkyl, aryl, aryl-lower alkyl, heteroaryl, lower alkoxy, hydroxy or oxo. Examples of heterocyclyl are pyrrolidinyl, oxazolidinyl, thiazolidinyl, piperidinyl, morpholinyl, piperazinyl, dioxolanyl, tetrahydro-furanyl and tetrahydropyranyl.

Acyl designates, for example, alkylcarbonyl, cycloalkylcarbonyl, arylcarbonyl, aryl-lower alkylcarbonyl, or heteroarylcarbonyl. Lower acyl is preferably lower alkylcarbonyl, in particular propionyl or acetyl.

Hydroxyalkyl is especially hydroxy-lower alkyl, preferably hydroxymethyl, 2-hydroxyethyl or 2-hydroxy-2-propyl.

Cyanoalkyl designates preferably cyanomethyl and cyanoethyl.

Haloalkyl is preferably fluoroalkyl, especially trifluoromethyl, 3,3,3-trifluoroethyl or pentafluoroethyl.

Halogen is fluorine, chlorine, bromine, or iodine, in particular fluorine or chlorine.

Lower alkoxy is especially methoxy, ethoxy, iso-propyloxy, or tert-butyloxy.

Arylalkyl includes aryl and alkyl as defined hereinbefore, and is e.g. benzyl, chlorobenzyl, methoxybenzyl, 1-phenethyl or 2-phenethyl.

Heteroarylalkyl includes heteroaryl and alkyl as defined hereinbefore, and is e.g. 2-, 3- or 4-pyridylmethyl, 1- or 2-pyrrolylmethyl, 2- or 3-thiophenylmethyl, 1-pyrazolylmethyl, or 1-imidazolylmethyl, or such compounds wherein heteraryl is substituted, e.g. by chloro or methyl.

In substituted amino, the substituents are preferably those mentioned as substituents hereinbefore. In particular, substituted amino is alkylamino, dialkylamino, optionally substituted arylamino, optionally substituted arylalkylamino, lower alkylcarbonylamino, lower alkoxycarbonylamino or optionally substituted carbamoylamino.

Salts of compounds of the formula (I) are in particular pharmaceutically acceptable salts.

Such pharmaceutically acceptable salts are formed, for example, in compounds of formula (I) containing an acid function, e.g. carboxylic acid function, with organic or inorganic cations. Suitable inorganic cations are, for example, alkali cations, such as lithium, sodium or potassium cations, or earth alkali cations, such as magnesium, calcium, strontium and barium cations, or metallic cations, e.g. aluminium or transition metal cations. Preferred inorganic cations are sodium, potassium, magnesium and calcium cations. Suitable organic cations are, for example, tetrasubstituted ammonium cations, for example tetramethylammonium, protonated tri-, di- and mono-substituted amines, or ammonium. Suitable cations are derived by protonation from primary, secondary or tertiary amines containing, for example, lower alkyl, hydroxy-lower alkyl or hydroxy-lower alkoxy-lower alkyl groups, e.g., 2-hydroxyethylammonium, 2-(2-hydroxyethoxy)ethyldimethyl-ammonium, diethylammonium, di(2-hydroxyethyl)ammonium, trimethylammonium, triethylammonium, 2-hydroxyethyldimethylammonium, or di(2-hydroxyethyl)methyl-ammonium, also from correspondingly substituted cyclic secondary and tertiary amines, e.g., N-methylpyrrolidinium, N-methylpiperidinium, N-methylmorpholinium, N-2-hydroxy-ethylpyrrolidinium, N-2-hydroxyethylpiperidinium, or N-2-hydroxyethylmorpholinium, and the like. Preferred organic cations are 2-hydroxyethylammonium, diethylammonium or di(2-hydroxyethyl)ammonium.

In compounds of formula (I) containing a basic nitrogen function, pharmaceutically acceptable slats are formed with organic or inorganic acids. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methyl-benzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid. For isolation or purification purposes it is also possible to use pharmaceutically unacceptable salts, for example picrates or perchlorates.

For therapeutic use, only pharmaceutically acceptable salts or free compounds are employed (where applicable in the form of pharmaceutical preparations), and these are therefore preferred.

In view of the close relationship between the novel compounds in free form and those in the form of their salts, any reference to the free compounds hereinbefore and hereinafter is to be understood as referring also to the corresponding salts, as appropriate and expedient. The compounds of formula (I), including their salts, are also obtainable in the form of hydrates, or their crystals can include, for example, the solvent used for crystallization, i.e. be present as solvates. Any reference to the free compounds hereinbefore and hereinafter is also to be understood as referring to the corresponding hydrates and solvates.

The compound of the formula (I) may be administered in the form of a pro-drug which is broken down in the human or animal body to give a compound of the formula (I). Examples of pro-drugs include in vivo hydrolysable esters and amides of a compound of the formula (I), for example esters or amides of naturally occurring α-amino acids or di- or tripeptides formed from naturally occurring α-amino acids.

Compounds of formula (I) are prepared by methods known in the art, in particular by condensation reactions of carboxylic acids or suitably activated acid derivatives, with amines or amine derivatives. If one or more other functional groups, for example carboxy, hydroxy or amino, are or need to be protected in starting compounds or intermediates, because they should not take part in the reaction, these are such protecting groups as are usually applied in the synthesis of amides, in particular peptide compounds. Particular syntheses schemes and reaction conditions are explained in detail in the Examples.

The compounds of formula (I) have valuable pharmacological properties. In particular, compounds of the invention and pharmaceutical compositions containing them are useful as neurotrypsin inhibitors.

Skeletal muscle atrophy is accompanied by a substantial loss of muscle strength and plays a major role in the pathogenesis of frailty and functional impairment that occurs with progressive old age. Weakness of the lower extremities has been implicated in a number of functional impairments, such as difficulties in rising from a chair or getting out of bed, slow speed of gait and other movements, and difficulties to maintain balance, resulting in falls and injuries. Skeletal muscle fiber loss has a negative effect on both the absolute strength that a muscle can develop and the speed with which a muscle can develop strength.

Increasing age is associated with a progressive decrease of the metabolic rate, which in turn has substantial physiological consequences, including a reduced tolerance against heat and cold as well as an increased propensity to develop obesity. Skeletal muscles comprise approximately 40% of the fat-free body mass and play an important homeostatic role in the body's metabolism. Therefore, a reduction of the skeletal muscle mass with increasing age is a major contributor to the decreased metabolic rate. By preventing the progressive fiber loss, the inhibition of neurotrypsin acts against these metabolic and physiological consequences.

Progressive loss of skeletal muscle mass and strength with age has been recognized as a major contributor to the gradual reduction of bone density observed with increasing age. Conversely, it is well known that the forces exerted on the bones by muscular activity stimulate bone formation. Thus, forces generated by muscle contraction are an important determinant of bone quality. Preventing muscle fiber loss by inhibition of peripheral neurotrypsin activity may therefore prevent or linder the adverse effects on skeletal muscle quality and indirectly antagonize progression of osteoporosis.

Beneficial effects of neurotrypsin inhibition may also be expected for skeletal muscle atrophies that occur in numerous clinical situations in which muscle wasting is an accompanying problem, including cancer, AIDS, and sepsis.

The concept of synapse tuning by reducing the anti-synaptic activity of neurotrypsin and, thereby, enhancing pro-synaptic activities at the expense of anti-synaptic activities, offers a wide range of applications in the area of disturbed cognitive brain functions. In particular, inhibition of neurotrypsin is beneficial in diseases and subclinical situations where synapse formation and the increase in the size and the strength of existing synapses is needed.

Inhibition of neurotrypsin is useful in the treatment of schizophrenia. Excessive neurotrypsin at the synapse drives synaptic pruning and, thus, generates a synaptic phenotype that is in accordance with the synaptic phenotype found in the brain of patients with schizophrenia. This experimental observation qualifies neurotrypsin as one of the factors that drive synaptic pruning. In a situation, where excessive synaptic pruning occurs due to the convergent action of multiple pruning-promoting factors, controlled and subtle partial inhibition of neurotrypsin diminishes the drive for synaptic pruning. This allows a recovery from the “schizophrenic synaptic phenotype” and results in the alleviation of the schizophrenic symptoms. The reduction of synapse numbers in the CNS of neurotrypsin-overexpressing mice indicates that inhibition of neurotrypsin results in a lesser degree of synaptic pruning and, thus, increased synaptic number and enhanced neuronal connectivity and communication. Compounds according to the invention inhibiting the enzymatic function of neurotrypsin are, therefore, useful in reverting the synaptic alterations in schizophrenia and in re-establishing normal synaptic structure and function and, thus, stop or shorten schizophrenic episodes and protect from new episodes.

Neurotrypsin is implicated in the development of cognitive disorders and mental retardation. A neurotrypsin gene knockout in humans causes mental retardation. Neurotrypsin gene duplications are implicated in the development of autism disorders. It is also probable that neurotrypsin plays a role in the development of Alzheimer disease (AD), as agrin fragments are found in senile plaques in AD patients (Van Horssen, J. et al., Acta Neuropathol. 2001, 102:604-14). It is well known that loss of lean muscular mass (i.e. sarcopenia) is accelerated especially in the earlier stages of Alzheimer disease (AD) and is associated with brain atrophy and cognitive impairment. A potential explanation for these observations is that AD and sarcopenia share common underlying pathogenic mechanisms (Burns et al., Arch Neurol. 2010, 67(4):428-433). It is well accepted that the dysfunction of acetylcholine containing neurons contributes substantially to the cognitive decline observed in those with advanced age and Alzheimer's disease (AD). This premise has since served as the basis for the majority of treatment strategies and drug development approaches for AD to date. Neurotrypsin is present at cholinergic synapses in the brain and in neuromuscular junctions. Over-production of neurotrypsin could contribute to the development and/or progression of both AD and sarcopenia.

Inhibition of neurotrypsin also supports cognitive enhancement in mild cognitive impairment and other clinical and subclinical states with reduced cognitive functions. Mild cognitive impairment, as well as other clinical and subclinical states of impaired cognitive functions have been found to be associated with evidence for cerebral tissue atrophy in several CNS areas. The reduction of synapse numbers in the CNS of neurotrypsin-overexpressing mice indicates that inhibition of neurotrypsin results in an increased synaptic number and enhanced neuronal connectivity and communication. Compounds according to the invention inhibiting the enzymatic function of neurotrypsin are, therefore, useful in reverting the synaptic alterations in all clinical and subclinical disorders in which a reduced number of synapses or a reduced function of synapses is involved, and in re-establishing normal synaptic structure and function. By this, pharmaceutical inhibition of neurotrypsin may improve cognitive functions in different states with reduced cognitive functions of heterogenous origins.

Based on these facts, the invention further relates to neurotrypsin inhibitors of formula (I) as described above and below for use in the treatment and/or prophylaxis of diseases caused by deficiency of synapses, for example skeletal muscle atrophy, schizophrenia and cognitive disturbance. Skeletal muscle atrophy to be treated is in particular so-called sarcopenia, i.e. a skeletal muscle atrophy due to old age, skeletal muscle atrophy accompanied by osteoporosis, and skeletal muscle atrophy due to muscle wasting associated with a severe disease, such as cancer, AIDS and sepsis, or also skeletal muscle atrophy as a consequence of immobilization and/or bed rest due to a severe injury or a severe disease. Schizophrenia to be treated is a disorder in the entire field of schizophrenia and schizophrenia-like disorders, comprising chronic schizophrenia, chronic schizo-affective disorders, unspecific disorders, acute and chronic schizophrenia of various symptomatologies, as for example severe, non-remitting “Kraepelinic” schizophrenia or the DSM-III-R-prototype of the schizophrenia-like disorders, episodic schizophrenic disorders, delusionic schizophrenia-like disorders, schizophrenia-like personality disorders, as for example schizophrenia-like personality disorders with mild symptomatics, schizotypic personality disorders, the latent forms of schizophrenic or schizophrenia-like disorders, and non-organic psychotic disorders. Furthermore, neurotrypsin inhibitors as described herein may be used as cognitive enhancers, for improving brain performance and for ameliorating learning and memory functions. Cognitive deficiencies to be treated are mild cognitive impairment, e.g. in a potential early stage of Alzheimer's disease, impairment of cognitive function without dementia in elderly people, and impairment of cognitive functions in patients with Alzheimer's disease, Parkinson's disease, multiple sclerosis, stroke, and head trauma.

Likewise the invention relates to the use of neurotrypsin inhibitors of formula (I) for the manufacture of a medicament for the treatment and/or prophylaxis of diseases caused by deficiency of synapses, as defined hereinbefore.

Furthermore the invention relates to the treatment and/or prophylaxis of diseases caused by deficiency of synapses, for example skeletal muscle atrophy, schizophrenia, Alzheimer disease and cognitive disturbance, which comprises administering a compound of formula (I) or a pharmaceutically acceptable salt thereof, in a quantity effective against said disease, to a warm-blooded animal requiring such treatment. The compounds of formula (I) can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.05 g to approximately 5 g, preferably from approximately 0.25 g to approximately 1.5 g, of a compound of the present invention.

A compound of formula (I) can be administered alone or in combination with one or more other therapeutic agents, possible combination therapy taking the form of fixed combinations, or the administration of a compound of the invention and one or more other therapeutic agents being staggered or given independently of one another, or the combined administration of fixed combinations and one or more other therapeutic agents.

The present invention relates also to pharmaceutical compositions that comprise a compound of formula (I) as active ingredient and that can be used especially in the treatment of the diseases mentioned hereinbefore. Compositions for enteral administration, such as nasal, buccal, rectal or, especially, oral administration, and for parenteral administration, such as intravenous, intramuscular or subcutaneous administration, to warm-blooded animals, especially humans, are especially preferred. The compositions comprise the active ingredient alone or, preferably, together with a pharmaceutically acceptable carrier. The dosage of the active ingredient depends upon the disease to be treated and upon the species, its age, weight, and individual condition, the individual pharmacokinetic data, and the mode of administration.

The present invention relates especially to pharmaceutical compositions that comprise a compound of formula (I), a tautomer, a prodrug or a pharmaceutically acceptable salt, or a hydrate or solvate thereof, and at least one pharmaceutically acceptable carrier.

The pharmaceutical compositions comprise from approximately 1% to approximately 95% active ingredient, single-dose administration forms comprising in the preferred embodiment from approximately 20% to approximately 90% active ingredient and forms that are not of single-dose type comprising in the preferred embodiment from approximately 5% to approximately 20% active ingredient. Unit dose forms are, for example, coated and uncoated tablets, ampoules, vials, suppositories, or capsules. Further dosage forms are, for example, ointments, creams, pastes, foams, tinctures, lip-sticks, drops, sprays, dispersions, etc. Examples are capsules containing from about 0.05 g to about 1.0 g active ingredient.

The pharmaceutical compositions of the present invention are prepared in a manner known per se, for example by means of conventional mixing, granulating, coating, dissolving or lyophilizing processes.

Preference is given to the use of solutions of the active ingredient, and also suspensions or dispersions, especially isotonic aqueous solutions, dispersions or suspensions which, for example in the case of lyophilized compositions comprising the active ingredient alone or together with a carrier, for example mannitol, can be made up before use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving and lyophilizing processes. The said solutions or suspensions may comprise viscosity-increasing agents, typically sodium carboxymethylcellulose, carboxymethylcellulose, dextran, polyvinylpyrrolidone, or gelatins, or also solubilizers, e.g. Tween 80® (polyoxyethylene(20)sorbitan mono-oleate).

Suspensions in oil comprise as the oil component the vegetable, synthetic, or semi-synthetic oils customary for injection purposes. In respect of such, special mention may be made of liquid fatty acid esters that contain as the acid component a long-chained fatty acid having from 8 to 22, especially from 12 to 22, carbon atoms. The alcohol component of these fatty acid esters has a maximum of 6 carbon atoms and is a monovalent or polyvalent, for example a mono-, di- or trivalent, alcohol, especially glycol and glycerol. As mixtures of fatty acid esters, vegetable oils such as cottonseed oil, almond oil, olive oil, castor oil, sesame oil, soybean oil and groundnut oil are especially useful.

The manufacture of injectable preparations is usually carried out under sterile conditions, as is the filling, for example, into ampoules or vials, and the sealing of the containers.

Suitable carriers are especially fillers, such as sugars, for example lactose, saccharose, mannitol or sorbitol, cellulose preparations, and/or calcium phosphates, for example tricalcium phosphate or calcium hydrogen phosphate, and also binders, such as starches, for example corn, wheat, rice or potato starch, methylcellulose, hydroxypropyl methyl-cellulose, sodium carboxymethylcellulose, and/or polyvinylpyrrolidone, and/or, if desired, disintegrators, such as the above-mentioned starches, also carboxymethyl starch, crosslinked polyvinylpyrrolidone, alginic acid or a salt thereof, such as sodium alginate. Additional excipients are especially flow conditioners and lubricants, for example silicic acid, talc, stearic acid or salts thereof, such as magnesium or calcium stearate, and/or polyethylene glycol, or derivatives thereof.

Tablet cores can be provided with suitable, optionally enteric, coatings through the use of, inter alia, concentrated sugar solutions which may comprise gum arabic, talc, polyvinyl-pyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropyl-methylcellulose phthalate. Dyes or pigments may be added to the tablets or tablet coatings, for example for identification purposes or to indicate different doses of active ingredient.

Pharmaceutical compositions for oral administration also include hard capsules consisting of gelatin, and also soft, sealed capsules consisting of gelatin and a plasticizer, such as glycerol or sorbitol. The hard capsules may contain the active ingredient in the form of granules, for example in admixture with fillers, such as corn starch, binders, and/or glidants, such as talc or magnesium stearate, and optionally stabilizers. In soft capsules, the active ingredient is preferably dissolved or suspended in suitable liquid excipients, such as fatty oils, paraffin oil or liquid polyethylene glycols or fatty acid esters of ethylene or propylene glycol, to which stabilizers and detergents, for example of the polyoxy-ethylene sorbitan fatty acid ester type, may also be added.

Pharmaceutical compositions suitable for rectal administration are, for example, suppositories that consist of a combination of the active ingredient and a suppository base. Suitable suppository bases are, for example, natural or synthetic triglycerides, paraffin hydrocarbons, polyethylene glycols or higher alkanols.

For parenteral administration, aqueous solutions of an active ingredient in water-soluble form, for example of a water-soluble salt, or aqueous injection suspensions that contain viscosity-increasing substances, for example sodium carboxymethylcellulose, sorbitol and/or dextran, and, if desired, stabilizers, are especially suitable. The active ingredient, optionally together with excipients, can also be in the form of a lyophilizate and can be made into a solution before parenteral administration by the addition of suitable solvents.

Solutions such as are used, for example, for parenteral administration can also be employed as infusion solutions.

Preferred preservatives are, for example, antioxidants, such as ascorbic acid, or microbicides, such as sorbic acid or benzoic acid.

With the groups of preferred compounds of formula (I) mentioned hereinafter, definitions of substituents from the general definitions mentioned hereinbefore may reasonably be used, for example, to replace more general definitions with more specific definitions or especially with definitions characterized as being preferred.

Preferred are compounds of formula (I) wherein

R¹ is optionally substituted biphenylyl, phenoxyphenyl or phenylaminophenyl, optionally substituted 1H-benzimidazol-2-yl-phenyl, optionally substituted benzo-C₅- or C₆-cycloalkyl or -cycloalkenyl, optionally substituted phenyl-thiophenyl or benzothiophenyl, optionally substituted 1H-benz[d]imidazol-2-yl, optionally substituted indolyl, optionally substituted quinolinyl, or optionally substituted phenyl-1,3-thiazol-2-yl or benzo-1,3-thiazol-2-yl;
R² is hydrogen or methyl;
R³ is alkyl, optionally substituted benzyl, optionally substituted phenylethyl, optionally substituted phenyl; and
R⁴ is hydrogen or lower alkyl; or
R³ and R⁴ together with the nitrogen atom, to which they are bound, are optionally substituted pyrrolidino, optionally substituted piperidino, 1,2,3,4-tetrahydro-quinol-1-yl or 1,2,3,4-tetrahydro-isoquinol-2-yl, morpholino, or optionally substituted piperazino.

More preferred are compounds of formula (I) wherein

R¹ is optionally substituted biphenylyl, phenoxyphenyl or phenylaminophenyl with one to three substituents, wherein the substituents are selected from the group consisting of lower alkyl, hydroxy, lower alkoxy, halo or cyano; 1H-benzimidazol-2-yl-phenyl optionally substituted at nitrogen by methyl or carboxymethyl and at the benzo residue by carboxy, chloro or dichloro;
2-indanyl or 1H-2-indenyl, optionally substituted by chloro and/or phenyl;
phenyl-thiophenyl, optionally substituted by lower alkyl, lower alkoxy, ethylenedioxy, halo or cyano;
benzo[b]thiophen-2- or -3-yl, optionally substituted by halo, dihalo or ethylenedioxy; 1H-benz[d]imidazol-2-yl, optionally substituted by phenyl, halo, dihalo, carboxy or methoxycarbonyl;
2- or 3-indolyl, optionally substituted by phenyl, lower alkyl, halo, or acetoxy;
2- or 3-quinolyl, optionally substituted by lower alkyl, halo, or acetoxy; or
phenyl-1,3-thiazol-2-yl, optionally substituted by lower alkyl, halo, or acetoxy, benzo-1,3-thiazol-2-yl or halobenzo-1,3-thiazol-2-yl;
R² is hydrogen;
R³ is alkyl, in particular lower alkyl;
optionally substituted phenyl with one to three substituents, wherein the substituents are selected from the group consisting of halo, cyano, lower alkyl, hydroxy-lower alkyl, phenyl-hydroxy-lower alkyl, optionally halogenated benzyl, methylamino-lower alkyl, dimethyl-amino-lower alkyl, carbamidoyl-lower alkyl, hydroxy, lower alkoxy, hydroxy-lower alkoxy, phenoxy, benzyloxy, pyridoxy, phenyl, carboxy, phenylcarbonyl, carbamimidoyl, methyl-sulfonyl, N,N-dimethylsulfamoyl, and N-carbamimidoylsulfamoyl; and
R⁴ is hydrogen or lower alkyl; or
R³ and R⁴ together with the nitrogen atom, to which they are bound, are pyrrolidino, optionally substituted by phenyl, chlorophenyl, benzyl or phenoxy; piperidino, optionally substituted by phenyl, chlorophenyl, benzyl or phenoxy; morpholino; or piperazino, optionally substituted by 4-lower alkyl or 4-benzyl.

Particularly preferred are compounds of formula (I) wherein

R¹ is optionally substituted biphenylyl, phenoxyphenyl or phenylaminophenyl, in particular halo- or dihalo-biphenylyl, halo- or dihalo-phenoxyphenyl or halo- or dihalo-phenylamino-phenyl; 4-(1H-benzimidazol-2-yl)phenyl or 4-(1H-benzimidazol-2-yl)-3-hydroxy-phenyl, wherein benzimidazolyl is optionally substituted at nitrogen by methyl or carboxymethyl and at the benzo residue by carboxy, chloro or dichloro, for example as 5-chloro-, 5-carboxy- or 5,6-dichloro-1H-benzimidazol-2-yl;
optionally substituted phenyl-thiophen-2-yl, in particular 3- or 5-phenyl-, 3- or 5-p-fluorophenyl-, 3- or 5-p-chlorophenyl- or 5-(3,4-ethylenedioxyphenyl)-thiophen-2-yl; optionally substituted benzo[b]thiophen-2-yl, in particular benzo[b]thiophen-2-yl, 3- or 6-chloro-, 3,6-dichloro- or 5,6-ethylenedioxy-3-chloro-benzo[b]thiophen-2-yl; optionally substituted 1H-benz[d]imidazol-2-yl, in particular 1H-benz[d]imidazol-2-yl, 5- or 6-chloro, carboxy or methoxycarbonyl-1H-benz[d]imidazol-2-yl or 5,6-dichloro-1H-benz[d]imidazol-2-yl;
optionally substituted 2-indolyl, in particular 2-indolyl, 3-phenyl-, 1-methyl-, 1-methyl-3-phenyl-, 5-chloro-, 5-chloro-3-phenyl-, 6-chloro- or 5,6-dichloro-2-indolyl; or
optionally substituted 2-quinolyl, in particular 2-quinolyl, 6- or 7-chloro- or 6,7-dichloro-2-quinolyl;
R² is hydrogen;
R³ is alkyl, in particular methyl, iso-propyl, iso-butyl or iso-pentyl;
optionally substituted phenyl, in particular 2-, 3- or 4-chlorophenyl, 2- or 4-fluorophenyl, 2-,
3- or 4-bromophenyl, 2-fluoro-4-chlorophenyl, 2,4-difluorophenyl, 3- or 4-cyanophenyl, 3-hydroxymethylphenyl, 3-hydroxymethyl-4-chloro-phenyl, 4-lower alkylphenyl, such as 4-methyl-, 4-iso-propyl, 4-n-butyl-, 4-iso-butyl- or 4-tert-butyl-phenyl, or 4-dimethylaminomethyl-phenyl; and
R⁴ is hydrogen, lower alkyl, in particular methyl, iso-propyl, iso-butyl or iso-pentyl; or R³ and R⁴ together with the nitrogen atom, to which they are bound, are optionally substituted pyrrolidino, in particular 3-phenyl, 3-p-chlorophenyl-, 3-benzyl- or 3-phenoxy-pyrrolidino, optionally substituted piperidino, in particular 3- or 4-phenoxy-piperidino, 4-phenyl-, 4-p-fluorophenyl-, 4-p-chlorophenyl-, 4-p-hydroxyphenyl-, 4-p-methoxyphenyl- or 4-(2,6-dimethylphenyl)-piperidino, 4-benzyl-, 4-p-fluorobenzyl-, 4-p-chlorobenzyl- or 4-p-hydroxybenzyl-piperidino; morpholino; or optionally substituted piperazino, in particular 4-benzyl- or 4-tert-butyl-piperazino.

Most preferred are compounds of formula (I) wherein

R¹ is p-biphenylyl, 4′-fluoro- or 4′-chloro-(p-biphenylyl), 4-phenoxyphenyl, 4-(m- or p-chlorophenoxy)phenyl, 4-(3,4-dichlorophenoxy)phenyl, 4-(1H-benzimidazol-2-yl)phenyl, (5-chloro- or 5,6-dichloro-1H-benzimidazol-2-yl)phenyl; 3- or 5-phenyl- or 5-p-fluorophenyl-thiophen-2-yl; benzo[b]thiophen-2-yl, 3- or 6-chloro-benzo[b]thiophen-2-yl; 2-indolyl, 5-chloro-3-phenyl-2-indanyl; 2-quinolyl, or 6- or 7-chloro- or 6,7-dichloro-2-quinolyl;
R² is hydrogen;
R³ is methyl, iso-propyl, or iso-butyl; 2-, 3- or 4-chlorophenyl, 2- or 4-fluorophenyl, 2-fluoro-4-chlorophenyl, 2,4-difluorophenyl, or 4-methyl-, 4-iso-propyl, 4-n-butyl-, 4-iso-butyl- or 4-tert-butyl-phenyl; and
R⁴ is hydrogen, methyl, iso-propyl, or iso-butyl;
or R³ and R⁴ together with the nitrogen atom, to which they are bound, are 4-phenoxy-piperidino, 4-phenyl-, 4-p-fluorophenyl- or 4-p-chlorophenyl-piperidino, or 4-benzyl-piperidino.

Also most preferred are the compounds of the Examples, in particular the compounds of Examples 1, 3, 6, 9, 11, 12, and 14.

### EXAMPLES

### Abbreviations

DCM=dichloromethane
DMF=dimethylformamide
eq.=equivalent
Et₂O=diethyl ether
EtOH=ethanol
h=hours
NMR=nuclear magnetic resonance
MeOH=methanol
min=minutes
MP=melting point
MS=mass spectrum
RT=room temperature
THF=tetrahydrofuran
TFA=trifluoroacetic acid
TMS=tetramethylsilane

¹H NMR spectra were recorded on Bruker AVANCE 400 MHz instrument. The ¹H chemical shifts are reported in ppm relative to TMS as an internal standard. Mass spectra were run on a MDS SCIEX-API-2000 instrument.

### Preparation 1: 4-Amino-N-(2-fluorophenyl)-2-hydroxybenzamide

A mixture of 2-hydroxy-4-nitrobenzoic acid (2.5 g, 13.65 mmol), SOCl₂ (1.993 ml, 27.3 mmol) and 1 drop of DMF in 100 ml of toluene is stirred at 90° C. for 6 h; a further amount of SOCl₂ (1 ml, 13.70 mmol) and 2 drops of DMF were added and stirring at 90° C. was continued for 1.5 h to complete the reaction. The solvent was evaporated under vacuum and the residue is used as such in the subsequent reaction.

A solution of 2-hydroxy-4-nitrobenzoyl chloride (393 mg, 1.95 mmol) in dry THF is added to a solution of 2-fluoroaniline (238 mg, 2.145 mmol) in 7-8 ml of dry THF, then pyridine (237 μl, 2.93 mmol) is added and the mixture is stirred overnight at RT. The desired product is detected in the LC-MS trace, but many impurities are also present. The mixture is diluted with AcOEt and washed with aq. dil. NaHCO₃ and twice with 1 N HCl, then with brine and finally dried and evaporated. The solid residue is triturated with a small amount of AcOEt and recovered by filtration, then washed again twice with hot AcOEt and twice with DCM. After drying, N-(2-fluorophenyl)-2-hydroxy-4-nitrobenzamide (114 mg, 0.413 mmol, 21.16% yield) is obtained.

A mixture of N-(2-fluorophenyl)-2-hydroxy-4-nitrobenzamide (114 mg, 0.413 mmol) and 10% Pd/C (65.9 mg, 0.062 mmol) in about 40 ml of AcOEt/MeOH ˜9:1 is hydrogenated in a Parr apparatus at 40 psi for 4 h: reaction complete. The mixture is filtered through celite and Na₂SO₄ and evaporated; the residue is washed with Et₂O/Hex and recovered by filtration: 4-amino-N-(2-fluorophenyl)-2-hydroxybenzamide (60 mg, 0.244 mmol, 59.0% yield) is obtained.

With the same method were also prepared:
4-amino-N-(4-chlorophenyl)-2-hydroxybenzamide
4-amino-N,N-diisobutyl-2-hydroxybenzamide
4-amino-N-(4-chloro-2-fluorophenyl)-2-hydroxybenzamide
4-amino-N-(2,4-difluorophenyl)-2-hydroxybenzamide
4-amino-N-(4-butylphenyl)-2-hydroxybenzamide

### Preparation 2: 4-Benzyl-1-(2-hydroxy-4-aminobenzoyl)piperidine

A solution of 2-hydroxy-4-nitrobenzoyl chloride (393 mg, 1.95 mmol) in dry THF is added to a solution of 4-benzylpiperidine (752 mg, 4.29 mmol) in 7-8 ml of dry THF, then the mixture is stirred overnight at RT, until the reaction is complete. The mixture is diluted with AcOEt and washed with aq. dil. NaHCO₃ and twice with 1 N HCl, then with brine and finally dried and evaporated. The solid residue is triturated with a small amount of Hex/DCM ˜7:3 and recovered by filtration, then washed again with the same mixture. After drying, 4-benzyl-1-(2-hydroxy-4-nitrobenzoyl)piperidine (239 mg, 0.702 mmol, 36.0% yield) is obtained.

A solution of this compound in about 40 ml of AcOEt/MeOH ˜9:1 is hydrogenated in a Parr apparatus at 35 psi for 7 h in the presence of Pd/C (127.4 mg, 0.119 mmol). The mixture is filtered through celite and Na₂SO₄ and evaporated; the residue is purified by prep. HPLC (neutral MeCN/water mobile phase) to obtain the title compound (182 mg, 0.586 mmol, 84% yield).

4-Phenoxy-1-(2-hydroxy-4-aminobenzoyl)piperidine was prepared similarly.

### Preparation 3: 2-Hydroxy-4-nitrobenzoyl chloride

A mixture of 2-hydroxy-4-nitrobenzoic acid (2.5 g, 13.65 mmol), SOCl₂ (1.993 ml, 27.3 mmol) and 1 drop of DMF in 100 ml of toluene is stirred at 90° C. for 6 h; a further amount of SOCl₂ (1 ml, 13.70 mmol) and 2 drops of DMF were added and stirring at 90° C. was continued for 1.5 h to complete the reaction. The solvent was evaporated under vacuum and the residue is used as such in the subsequent reaction.

### Preparation 4: 4′-Fluorobiphenyl-4-carbonyl chloride

A mixture of 4-bromobenzoic acid (300 mg, 1.492 mmol), 4-fluorophenylboronic acid (251 mg, 1.791 mmol), potassium fluoride (217 mg, 3.73 mmol) and Pd(OAc)₂ (33.5 mg, 0.149 mmol) in 10 ml of MeOH is microwave-heated at 110° C. for 2 h. After cooling, the precipitate is recovered by filtration, dissolved in AcOEt and washed with water and brine; after drying and evaporating, 4′-fluorobiphenyl-4-carboxylic acid (263 mg, 1.216 mmol, 82% yield) is obtained.

A mixture of 4′-fluorobiphenyl-4-carboxylic acid (180 mg, 0.833 mmol), 1 drop of DMF and (COCl)₂ (182 μl, 2.081 mmol) in 10 ml of dry DCM is stirred at RT for 5 h, until the conversion is complete. The mixture is evaporated completely; the residue is dissolved in 10 ml of anhydrous THF and used as such in the subsequent reaction.

### Preparation 5: 5-(4-Fluorophenyl)thiophene-2-carbonyl chloride

A mixture of 4-fluorophenylboronic acid (214 mg, 1.531 mmol), ethyl 5-bromothiophene-2-carboxylate (300 mg, 1.276 mmol), potassium fluoride (185 mg, 3.19 mmol) and Pd(OAc)₂ (28.6 mg, 0.128 mmol) in 10 ml of EtOH is microwave-heated at 110° C. for 2 h, until the reaction is complete. The mixture is diluted with AcOEt and washed with water and brine, then dried and evaporated; the residue is purified by flash chromatography (Hexane/Et₂O 95:5 to 9:1) to obtain ethyl 5-(4-fluorophenyl)thiophene-2-carboxylate (217 mg, 0.867 mmol, 67.9% yield).

A mixture of ethyl 5-(4-fluorophenyl)thiophene-2-carboxylate (217 mg, 0.867 mmol) and 6 N NaOH (433 μl, 2.60 mmol) in 15 ml of EtOH is stirred at 70° C. for 4 h. After cooling, the mixture is acidified and evaporated to dryness under vacuum; the residue is dissolved in AcOEt and washed with water and brine, then dried and evaporated to obtain 5-(4-fluorophenyl)thiophene-2-carboxylic acid (124 mg, 0.558 mmol, 64.4% yield).

A mixture of 5-(4-fluorophenyl)thiophene-2-carboxylic acid (124 mg, 0.558 mmol), 1 drop of DMF and (COCl)₂ (122 μl, 1.395 mmol) in 10 ml of dry DCM is stirred at RT for 5 h. The mixture is evaporated to dryness under vacuum; the residue is dissolved in 10 ml of anhydrous THF and used as such in the subsequent reaction.

### Preparation 6: 4-(3,4-Dichlorophenoxy)benzoyl chloride

A mixture of 3,4-dichlorophenol (300 mg, 1.840 mmol), ethyl 4-fluorobenzoate (310 mg, 1.840 mmol) and Cs₂CO₃ (660 mg, 2.025 mmol) in 15 ml of DMF is stirred overnight at 120° C. The cooled mixture is poured into ice/water, then extracted twice with AcOEt; the combined organic phases are washed 3 times with water, brine and finally dried and evaporated. The residue is purified by flash chromatography to obtain ethyl 4-(3,4-dichlorophenoxy)benzoate (175 mg, 0.562 mmol, 30.6% yield).

A mixture of ethyl 4-(3,4-dichlorophenoxy)benzoate (175 mg, 0.562 mmol) and 6 N NaOH (187 μl, 1.125 mmol) in 8 ml of EtOH is stirred overnight at RT, until the reaction is complete. The mixture is acidified and evaporated almost completely; the residue is diluted with water and the precipitate is collected by filtration and washed with water and a little amount of Et₂O/Hexanes. After drying, 4-(3,4-dichlorophenoxy)benzoic acid (128 mg, 0.452 mmol, 80% yield) is obtained.

A mixture of 4-(3,4-dichlorophenoxy)benzoic acid (128 mg, 0.452 mmol), 1 drop of DMF and (COCl)₂ (99 μl, 1.130 mmol) in 10 ml of dry DCM is stirred at RT for 5 h, until the conversion is complete. The mixture is evaporated to dryness under vacuum; the residue is dissolved in 10 ml of anhydrous THF and used as such in the subsequent reaction.

### Preparation 7: 6,7-Dichloroquinoline-2-carbonyl chloride

A mixture of 6,7-dichloro-2-methylquinoline (231 mg, 1.089 mmol), benzaldehyde (270 μl, 2.67 mmol) and Ac₂O (51.4 μl, 0.545 mmol) without any solvent is microwave-heated at 180° C. for 1 h. The mixture is triturated in water/EtOH ˜9:1 and the resulting solid is recovered by filtration and washed with Et₂O and Hexanes. After drying, clean (E)-6,7-dichloro-2-styrylquinoline (203 mg, 0.676 mmol, 62.1% yield) is obtained.

To a solution of (E)-6,7-dichloro-2-styrylquinoline (201 mg, 0.670 mmol) in 8 ml of pyridine and 2.5 ml of water cooled to 5° C., KMnO₄ (222 mg, 1.406 mmol) is added in portion while keeping the temperature under 10° C. After vigorously stirring for about 1 h, reaction is complete; the mixture is filtered through celite to eliminate Mn oxides and the pad is further washed with 2 N NaOH; the resulting solution is evaporated; the residue is acidified with aq. HCl and extracted with AcOEt, which is then washed with water, brine and finally dried and evaporated; the resulting solid is washed with DCM to obtain pure 6,7-dichloroquinoline-2-carboxylic acid (112 mg, 0.463 mmol, 69.1% yield).

A mixture of 6,7-dichloroquinoline-2-carboxylic acid (112 mg, 0.463 mmol), 1 drop of DMF and (COCl)₂ (101 μl, 1.157 mmol) in 10 ml of dry DCM is stirred at RT for 20 min, until the conversion is complete. The mixture is evaporated completely; the residue is dissolved in 10 ml of anhydrous THF and used as such in the subsequent reaction.

### Preparation 8: 4-(5,6-Dichloro-1H-benzo[d]imidazol-2-yl)benzoyl chloride

In a 1 liter Erlenmeyer flask, 4-formylbenzoic acid (10 g, 66.6 mmol) was dissolved in 400 ml of EtOH, then a solution of NaHSO₃ (6.93 g, 66.6 mmol) in 45 ml of water was added and the mixture was stirred while cooling in an ice bath for about 1 h. The white precipitate was collected and washed with a little amount of EtOH. After drying, 15.8 g of sodium (4-carboxyphenyl)(hydroxy)methanesulfonate (62.3 mmol, 94% yield) were obtained. In a 250 ml round-bottomed flask equipped with a condenser, a mixture of sodium (4-carboxyphenyl)(hydroxy)methanesulfonate (6.77 g, 26.6 mmol) and 4,5-dichlorobenzene-1,2-diamine (4.71 g, 26.6 mmol) in 60 ml of DMF was stirred at 170° C. overnight. The mixture was cooled to RT, poured into ice and acidified with 2 N HCl. The black precipitate was collected by filtration. The solid was stirred in 400 ml of methanol and the insoluble tarry portion was discarded by filtration through a Celite pad. The solvent was evaporated under vacuum and the resulting dark brown solid was washed with DCM to obtain 7.0 g of pure 4-(5,6-dichloro-1H-benzo[d]imidazol-2-yl)benzoic acid (22.79 mmol, 86% yield) as a pale brown solid.

4-(5,6-Dichloro-1H-benzo[d]imidazol-2-yl)benzoic acid (1.0 g, 3.26 mmol) was suspended in 18 ml of thionyl chloride. One drop of DMF was also added and the reaction was stirred at 70° C. overnight. The volatile fractions were removed under vacuum and the pale yellow 4-(5,6-dichloro-1H-benzo[d]imidazol-2-yl)benzoyl chloride (1.060 g, 3.26 mmol, 100% yield) thus obtained was used in the following step without further purification.

### Preparation 9: 5-Chloro-3-phenyl-1H-indole-2-carbonyl chloride

A mixture of ethyl 5-chloro-3-phenyl-1H-indole-2-carboxylate (697 mg, 2.325 mmol) and 6 N NaOH (775 μl, 4.65 mmol) in 25 ml of EtOH is stirred at 70° C. for 3 h, until the reaction is complete; the mixture is acidified and evaporated almost completely. More water is added and the precipitate is collected by filtration and dried in a vacuum oven. 5-Chloro-3-phenyl-1H-indole-2-carboxylic acid (620 mg, 2.282 mmol, 98% yield) is obtained as an orange solid.

To a suspension of 5-chloro-3-phenyl-1H-indole-2-carboxylic acid (250 mg, 0.920 mmol) in 10 ml of DCM, one small drop of DMF is added, followed by (COCl)₂ (161 μl, 1.840 mmol); the mixture is stirred at RT for 3.5 h, until the reaction is complete (sample quenched with MeOH). The mixture is completely evaporated at low temperature and the residue is used as such for the following reactions.

### Example 1

### N-(4-Chlorophenyl)-4-(4-(5,6-dichloro-1H-benzo[d]imidazol-2-yl)benzamido)-2-hydroxybenzamide

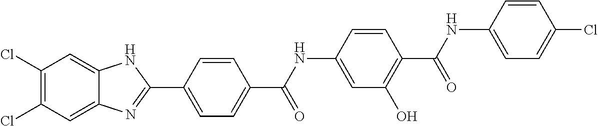 [SMILES: O=C(Nc1ccc(C(=O)Nc2ccc(Cl)cc2)c(O)c1)c1ccc(-c2nc3cc(Cl)c(Cl)cc3[nH]2)cc1]

A mixture of 4-(5,6-dichloro-1H-benzo[d]imidazol-2-yl)benzoyl chloride (0.840 g, 2.58 mmol) and 4-amino-N-(4-chlorophenyl)-2-hydroxybenzamide (1.355 g, 5.16 mmol) in 100 ml of dry THF is stirred overnight at RT, until the reaction is complete. A few ml of water are added, then the mixture is evaporated under vacuum; the residue is stirred in 120 ml of methanol containing a few ml of water for 2 h, then recovered by filtration and washed thoroughly with methanol; after drying in a vacuum oven, crude N-(4-chlorophenyl)-4-(4-(5,6-dichloro-1H-benzo[d]imidazol-2-yl)benzamido)-2-hydroxybenzamide (1.09 g, 1.975 mmol, 77% yield) is obtained as a yellow solid.

The crude compound is suspended in 100 ml of MeOH/DMSO-95:5 and stirred at reflux for about 2 h. After cooling, the precipitate is collected by suction filtration and washed with water and methanol. After drying in a vacuum oven, pure N-(4-chlorophenyl)-4-(4-(5,6-dichloro-1H-benzo[d]imidazol-2-yl)benzamido)-2-hydroxybenzamide (0.85 g, 78%) is obtained, mp>220° C.

¹H-NMR (300 MHz, DMSO-d₆, 300° K), δ ppm: 13.43 (br.s, 1H), 11.99 (s, 1H), 10.56 (s, 1H), 10.38 (s, 1H), 8.28-8.41 (m, 2H), 8.10-8.23 (m, 2H), 7.99 (d, 1H), 7.97 (br.s, 1H), 7.84 (br.s, 1H), 7.72-7.81 (m, 2H), 7.67 (d, 1H), 7.42-7.47 (m, 2H), 7.39 (dd, 1H)

MS: m/z=550.9 [M-H]⁺

Analogously the compounds of the following examples were prepared:

| Ex. No. | Chemical name | mp ° C. | ¹H-NMR (300 MHz, DMSO-d₆, 300° K), δ ppm | MS, m/z |
|---|---|---|---|---|
| 2 | N-(2-Fluorophenyl)-4- | >220 | 12.19 (s, 1H) 11.99 (s, 1H) 10.57 (br. s, | 500.16 |
| | (5-chloro-3-phenyl- | | 1H) 10.04 (s, 1H) 8.22 (td, 1H) 7.96 (d, | [MH]+ |
| | indole-2- | | 1H) 7.44-7.67 (m, 7H) 7.14-7.43 (m, 5H) | |
| | carbonyl)amino-2- | | 7.04 (dd, 1H) | |
| | hydroxybenzamide | | | |
| 3 | N-(4-Chlorophenyl)-4- | >220 | 11.99 (s, 1H), 10.47 (s, 1H), 10.37 (s, | 461.11 |
| | (4-fluorophenyl)- | | 1H), 8.03-8.13 (m, 2H), 7.99 (d, 1H), | [MH]⁺ |
| | benzamido)-2- | | 7.79-7.89 (m, 4H), 7.72-7.79 (m, 2H), | |
| | hydroxybenzamide | | 7.66 (d, 1H), 7.41-7.48 (m, 2H), 7.25- | |
| | | | 7.41 (m, 3H) | |
| 4 | N-(4-Chlorophenyl)-4- | >220 | 11.98 (s, 1H), 10.74 (5, 1H), 10.40 (s, | 457.08 |
| | [(3-chlorobenzo[b]- | | 1H), 8.10-8.25 (m, 1H), 7.99 (d, 1H), | [MH]⁺ |
| | thiophene-2- | | 7.91-7.99 (m, 1H), 7.71-7.80 (m, 2H), | |
| | carbonyl)amino]-2- | | 7.60-7.69 (m, 2H), 7.54 (d, 1H), 7.39- | |
| | hydroxybenzamide | | 7.49 (m, 2H), 7.28 (dd, 1H) | |
| 5 | N-(4-Chlorophenyl)-4- | >220 | 11.97 (s, 1H), 10.40 (5, 1H), 10.36 (s, | 527.03 |
| | (3,4-dichlorophenoxy)- | | 1H), 8.00-8.10 (m, 2H), 7.97 (d, 1H), | [MH]⁺ |
| | benzamido)-2- | | 7.72-7.79 (m, 2H), 7.70 (d, 1H), 7.63 (d, | |
| | hydroxybenzamide | | 1H), 7.39-7.47 (m, 3H), 7.35 (dd, 1H), | |
| | | | 7.18-7.27 (m, 2H), 7.13 (dd, 1H) | |
| 6 | N-(4-Chlorophenyl)-5- | >220 | 11.98 (s, 1H), 10.39 (5, 1H), 10.37 (s, | 467.07 |
| | (4-fluorophenyl)- | | 1H), 8.07 (d, 1H), 7.98 (d, 1H), 7.78- | [MH]⁺ |
| | thiophene-2- | | 7.85 (m, 2H), 7.70-7.78 (m, 2H), 7.62 | |
| | carbonyl)amino-2- | | (d, 1H), 7.58 (d, 1H), 7.38-7.47 (m, 2H), | |
| | hydroxybenzamide | | 7.27-7.38 (m, 3H) | |
| 7 | N-(4-Chlorophenyl)-4- | >220 | [353° K] 11.85 (s, 1H), 10.67 (s, 1H), | 486.03 |
| | (6,7-dichloroquinoline- | | 10.29 (br. s, 1H), 8.65 (d, 1H), 8.50 (s, | [MH]⁺ |
| | 2-carbonyl)amino-2- | | 1H), 8.48 (s, 1H), 8.30 (d, 1H), 8.04 (d, | |
| | hydroxybenzamide | | 1H), 7.73-7.78 (m, 2H), 7.71 (d, 1H), | |
| | | | 7.45 (dd, 1H), 7.37-7.43 (m, 2H) | |
| 8 | N-(4-Chlorophenyl)-4- | >220 | 12.19 (5, 1H) 11.93 (5, 1H) 10.36 (br. s., | 516.13 |
| | (5-chloro-3- | | 1H) 9.99 (s, 1H) 7.91 (d, 1H) 7.62-7.82 | [MH]⁺ |
| | phenylindole-2- | | (m, 2H) 7.35-7.61 (m, 10H) 7.32 (dd, | |
| | carbonyl)amino-2- | | 1H) 7.02 (dd, 1H) | |
| | hydroxybenzamide | | | |
| 9 | N-(2-Fluoro-4-chloro- | >220 | 12.03 (br. s, 1H), 10.65 (d, 1H), 10.60 (s, | 569.04 |
| | phenyl)-4-(4-(5,6-di- | | 1H), 8.32-8.37 (m, 2H), 8.29 (t, 1H), | [MH]⁺ |
| | chloro-1H- | | 8.10-8.22 (m, 2H), 8.01 (d, 1H), 7.92 (s, | |
| | benzo[d]imidazol-2-yl)- | | 2H), 7.78 (d, 1H), 7.56 (dd, 1H), 7.40 | |
| | benzamido)-2- | | (dd, 1H), 7.33 (ddd, 1H) | |
| | hydroxybenzamide | | | |
| 10 | 1-(2-Hydroxy-4-(5,6- | >220 | [353° K + TFA] 10.11 (s, 1H), 8.30 (m, | 599.23 |
| | dichloro-1H- | | 2H), 8.14 (m, 2H), 7.86 (s, 2H), 7.51 (d, | [MH]⁺ |
| | benzo[d]imidazol-2-yl)- | | 1H), 7.13-7.37 (m, 6H), 7.10 (d, 1H), | |
| | benzoyl)-4- | | 3.82-4.16 (m, 2H), 2.76-2.99 (m, 2H), | |
| | benzylpiperidine | | 2.58 (d, 2H), 1.84 (m, 1H), 1.64 (dd, | |
| | | | 2H), 1.09-1.32 (m, 2H) | |
| 11 | N-(2,4-Difluorophenyl)- | >220 | 12.05 (br. s, 1H), 10.59 (s, 1H), 10.52 (s, | 553.06 |
| | 4-(4-(5,6-dichloro-1H- | | 1H), 8.26-8.44 (m, 2H), 8.07-8.23 (m, | [MH]⁺ |
| | benzo[d]imidazol-2-yl)- | | 2H), 8.07-8.23 (m, 1H), 8.02 (d, 1H), | |
| | benzamido)-2- | | 7.92 (s, 2H), 7.75 (d, 1H), 7.26-7.52 | |
| | hydroxybenzamide | | (m, 2H), 6.95-7.25 (m, 1H) | |
| 12 | 1-(2-Hydroxy-4-(5,6- | >220 | 10.39 (s, 1H), 9.90 (br. s, 1H), 8.27-8.39 | 601.31 |
| | dichloro-1H- | | (m, 2H), 8.06-8.20 (m, 2H), 7.92 (s, 2H), | [MH]⁺ |
| | benzo[d]imidazol-2-yl)- | | 7.59 (d, 1H), 7.25-7.34 (m, 2H), 7.23 | |
| | benzoyl)-4- | | (dd, 1H), 7.13 (d, 1H), 6.96-7.03 (m, | |
| | phenoxypiperidine | | 2H), 6.87-6.96 (m, 1H), 4.58-4.73 (m, | |
| | | | 1H), 3.76 (br. s, 2H), 3.13-3.43 (m, 2H), | |
| | | | 1.84-2.08 (m, 2H), 1.49-1.75 (m, 2H) | |
| 13 | N,N-Diisobutyl-2- | >220 | 10.36 (s, 1H), 9.82 (br. s, 1H), 8.33 (m, | 553.32 |
| | hydroxy-4-((5,6- | | 2H), 8.14 (m, 2H), 7.92 (s, 2H), 7.57 (d, | [MH]⁺ |
| | dichloro-1H- | | 1H), 7.21 (dd, 1H), 7.06 (d, 1H), 3.15- | |
| | benzo[d]imidazol-2-yl)- | | 3.41 (m, 2H), 2.88-3.15 (m, 2H), 1.92- | |
| | benzamido)benzamide | | 2.21 (m, 1H), 1.63-1.92 (m, 1H), 0.80- | |
| | | | 1.12 (m, 6H), 0.50-0.80 (m, 6H) | |
| 14 | N-(4-Butylphenyl)-4-(4- | >220 | 12.18 (br. s, 1H), 10.55 (s, 1H), 10.24 (s, | 573.3 |
| | (5,6-dichloro-1H- | | 1H), 8.27-8.42 (m, 2H), 8.11-8.22 (m, | [MH]⁺ |
| | benzo[d]imidazol-2-yl)- | | 2H), 8.02 (d, 1H), 7.92 (s, 2H), 7.64 (d, | |
| | benzamido)-2- | | H), 7.55-7.62 (m, 2H), 7.39 (dd, 1H), | |
| | hydroxybenzamide | | 7.14-7.24 (m, 2H), 2.57 (t, 2H), 1.46- | |
| | | | 1.66 (m, 2H), 1.32 (sxt, 2H), 0.91 (t, 3H) | |

### Determination of IC₅₀ for Human Neurotrypsin

Assessment of the potency of compounds of examples 1-14 in inhibiting the enzymatic activity of human neurotrypsin was performed according to the method described in patent application WO 2006/1103261

### Determination of the IC₅₀ for Various Proteases:

A 6 mM solution of the test compound in DMSO was prepared. In a V-bottom micro plate the 6 mM compound solution was diluted 6 times in DMSO to obtain a concentration row with the following concentrations: 6 mM, 1.8 mM, 540 μM, 162 μM, 48.8 μM, 14.4 μM, and 4 μM. From this DMSO master plate 1:20 dilutions in 10 mM MOPS pH 8.3 were prepared to obtain solutions of 300, 90, 27, 8.1, 2.44, 0.72 and 0.22 μM in MOPS buffer and 5% DMSO.

100 μl of this solution were pipetted into a flat bottom 96 well microplate and placed into a Tecan infinite 200 plate reader. Before measurement, 50 μl of protease solution and, subsequently 50 μl substrate solution were added by the machine. The liberation of free para nitroanilide was detected at 405 nm. The protease solution was freshly prepared by diluting to the desired concentration from a stock solution in 60 mM MOPS; 600 mM NaCl; 20 mM CaCl₂; 10% DMSO; 0.4% PEG 6000 pH 8.3. The substrate solution was prepared by dissolving the solid substrate in an adequate amount of water to get the desired concentrations. The used protease and substrate solutions are given below:

| Protease | concentration | Substrate | concentration |
|---|---|---|---|
| Trypsin | 2 nM | S-2222 | 0.5 mM |
| Thrombin | 6 nM | S-2266 | 0.5 mM |
| Plasmin | 8 nM | S-2288 | 0.5 mM |
| Kallikrein | 2 nM | S-2288 | 0.5 mM |
| tPA (2 chain) | 40 nM | S-2288 | 0.5 mM |
| uPA | 40 nM | S-2288 | 0.5 mM |
| Factor Xa | 5 nM | S-2288 | 0.5 mM |

All enzymes except trypsin were from human origin (American diagnostic). Trypsin was hog trypsin (Sigma Aldrich). Substrates were purchased from Chromogenix. S-2222 is N-Benzoyl-L-isoleucyl-L-glutamylglycyl-L-arginine-p-nitroaniline hydrochloride and its methyl ester (Phe-CO-Ile-Glu-(-OR)-Gly-Arg-pNA.HCl; 50% where R is H and 50% where R is CH3), S-2266 is H-D-Valyl-L-leucyl-L-arginine-p-Nitroaniline dihydrochloride (H-D-Val-Leu-Arg-pNA.2HCl) and S-2288 is H-D-Isoleucyl-L-prolyl-L-arginine-p-nitroaniline dihydrochloride (H-D-Ile-Pro-Arg-pNA.2HCl). The reactions were performed for 20-30 min and the initial velocities were plotted against the concentrations of the test compound. To obtain the IC₅₀, a curve with the equation

$$y = \frac{100}{1 + {( \frac{{IC}_{50}}{[ I ]} )}^{h}}$$

was fit to the data points, where “I” is the concentration of the test compound and h represents the Hill coefficient. The resulting IC₅₀ values for the compound of Example 1 are summarized below:

| IC₅₀ (μM) | Mean ± SD |
|---|---|
| Neurotrypsin | 0.47 ± 0.15 |
| Trypsin | >150 |
| Thrombin | >150 |
| Plasmin | >150 |
| Kallikrein | >150 |
| tPA | >150 |
| uPA | >150 |
| Factor Xa | 84.8 ± 46.44 |

### Test of In Vivo Efficacy of Compound of Example 1 on Body Weight and Plasma Levels of C-Terminal Agrin Fragment (CAF)

### Experimental Animals

Adult (P50) C57/BL6 mice from Charles River were used for the experiments. All animal experiments are performed in accordance with international guidelines for proper conduct of animal experiment under jurisdiction of the Swiss law (Permit number ZH-18/2008). For every mouse in the experiment the well-being during the interventions is documented in a score sheet.

The mice are housed under optimal hygienic conditions (OHB) with controlled air-conditioning and lighting (temperature: 20-24° C., humidity: 55-65%, day/night cycle: 12 h/12 h, lights on 7 a.m.). Individually ventilated cages of type II, L (530 cm² floor space) with standard litter are used. A maximum of 6 mice is kept in each cage. Mice are fed with standard rodent chow (Extrudat, KLIBA NAFAG) ad libitum and have free access to drinking water. Animal colony is managed and maintained at BioSupport, Schlieren.

### Treatment Schedule

Three adult mice were treated with 2.5 ml/kg of a 10 mg/ml solution of the compound of Example 1 in 100% DMSO. This corresponds to a dose of 25 mg/kg active ingredient. As vehicle control, 3 mice were treated with 2.5 ml/kg of 100% DMSO. Each mouse received three subcutaneous injections. Between dose 1 and dose 2 there were 8 h, between dose 2 and dose 3 16 h. 5 h after the last injection the mice were euthanatized with CO₂.

### Sample Collection and Preparation

Before the first injection the mice were weighted and ˜100 μl of blood was collected from the lateral tail vein. Serum was prepared with centrifugation of blood samples in Microvette 500 Z-Gel tubes (10,000 g, 20° C., 5 min). Supernatant was transferred to an Eppendorf reaction tube and centrifuged again (21,000 g, 20° C., 5 min). Supernatant was transferred to screw top reaction tubes and the serum was stored at −20° C. until analysis.

### Effect of Compound on Body Weight

For every animal the weight loss caused by treatment was computed as ratio of post-treatment/pre-treatment weight. The values were averaged for the compound of Example 1 and the vehicle treated mice, respectively. Sample means are indicated with standard deviations.

Weight loss in both groups of mice is below the 10% threshold. Compound of Example 1 did not produce any increase in weight loss compared to the vehicle control.

| | | Average weight loss | |
|---|---|---|---|
| Treatment | n | (% of T = 0 weigth) | SD |
| Vehicle | 3 | 4.99 | 2.39 |
| Example 1 | 3 | 3.99 | 0.68 |

### Analysis of Serum CAF Levels by Western Blotting

For the western blot analysis the blood samples from the two groups (vehicle and Example 1) and two sampling times (0 and 1) were pooled (10 μl of each individual sample) resulting in following two pools: Vehicle and Example 1. 50 μl of PBS was added to each pool and the diluted samples were centrifuged with 100 kDa size cut-off filters (Microcon, 14,000 g, RT, 30 min). Filtrates were mixed with 4×Lämmli buffer and loaded with a Hamilton syringe onto a 4-12% gradient NUPAGE gel for electrophoresis. Pooled sera from wild type mice and pooled sera from neurotrypsin knockout mice that had been processed as described above were loaded as controls. After electrophoresis the proteins were transferred onto a PVDF membrane with semi-dry blotting. The membrane was then stained with affinity purified (against recombinant C-terminal agrin fragment) polyclonal agrin antibodies. A secondary antibody conjugated with HRP together with the luminol/peroxide chemiluminescence system (ChemiGlow West, Alpha Innotech) was used to detect the CAF signal.

After staining for CAF the membrane was washed thoroughly and reused for a staining for endogenous mouse antibodies that served as loading control. A rabbit anti-mouse antibody conjugated with HRP together with the chemiluminiscence system described above was used. After imaging with a Stella imaging system (Raytest) the intensity of the CAF bands was measured with AIDA picture analysis software (Raytest) and normalized to the loading levels determined from mouse antibody light chains. The background present in the KO sample was then subtracted from every sample. The western blot was repeated once, the values for the CAF level were averaged and then normalized to the vehicle level. The normalized means with the standard deviation are depicted below. Treatment with compound of Example 1 led to reduction of CAF levels to 66.6%±12.9% of vehicle control.

| | Average CAF levels | |
|---|---|---|
| Treatment | % of vehicle | SD |
| Vehicle (DMSO) | 100.0 | 3.6 |
| Example 1 | 66.6 | 12.9 |

IC₅₀ was measured for all 14 examples with respect of neurotrypsin, and the results collected in the following Table:

| Example N° | Neurotrypsin IC₅₀, μM |
|---|---|
| 1 | 0.4 |
| 2 | 32.8 |
| 3 | 7.6 |
| 4 | 55.5 |
| 5 | 13.9 |
| 6 | 3.4 |
| 7 | 26.7 |
| 8 | 10.8 |
| 9 | 1.9 |
| 10 | 18.3 |
| 11 | 1.1 |
| 12 | 1.5 |
| 13 | 55.1 |
| 14 | 1.7 |

1. A compound of formula (I) 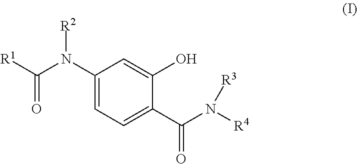
wherein
R¹ is phenyl substituted by phenyl, phenoxy, phenylamino or heteroaryl, all optionally further substituted; bicyclic aryl, monocyclic heteroaryl substituted by optionally substituted phenyl, or bicyclic heteroaryl;
R² is hydrogen or methyl;
R³ is alkyl, optionally substituted amino- or hydroxy-alkyl; aryl-lower alkyl, or aryl; and
R⁴ is hydrogen, lower alkyl, carboxy-, lower alkoxycarbonyl-, dimethylcarbamoyl-, hydroxy- or lower alkoxy-lower alkyl; or
R³ and R⁴ together with the nitrogen atom, to which they are bound, are optionally substituted pyrrolidino, optionally substituted piperidino, tetrahydro-quinolyl or -isoquinolyl, morpholino, or optionally substituted piperazino.

2. The compound according to claim 1 of formula (I) wherein
R¹ is optionally substituted biphenylyl, phenoxyphenyl or phenylaminophenyl, optionally substituted 1H-benzimidazol-2-yl-phenyl, optionally substituted benzo-C₅- or C₆-cycloalkyl or -cycloalkenyl, optionally substituted phenyl-thiophenyl or benzothiophenyl, optionally substituted 1H-benz[d]imidazol-2-yl, optionally substituted indolyl, optionally substituted quinolinyl, or optionally substituted phenyl-1,3-thiazol-2-yl or benzo-1,3-thiazol-2-yl;
R² is hydrogen or methyl;
R³ is alkyl, optionally substituted benzyl, optionally substituted phenylethyl, optionally substituted phenyl; and
R⁴ is hydrogen or lower alkyl; or
R³ and R⁴ together with the nitrogen atom, to which they are bound, are optionally substituted pyrrolidino, optionally substituted piperidino, 1,2,3,4-tetrahydro-quinol-1-yl or 1,2,3,4-tetrahydro-isoquinol-2-yl, morpholino, or optionally substituted piperazino.

3. The compound according to claim 1 of formula (I) wherein
R¹ is optionally substituted biphenylyl, phenoxyphenyl or phenylaminophenyl with one to three substituents, wherein the substituents are selected from the group consisting of lower alkyl, hydroxy, lower alkoxy, halo or cyano; 1H-benzimidazol-2-yl-phenyl optionally substituted at nitrogen by methyl or carboxymethyl and at the benzo residue by carboxy, chloro or dichloro; 2-indanyl or 1H-2-indenyl, optionally substituted by chloro and/or phenyl;
phenyl-thiophenyl, optionally substituted by lower alkyl, lower alkoxy, ethylenedioxy, halo or cyano;
benzo[b]thiophen-2- or -3-yl, optionally substituted by halo, dihalo or ethylenedioxy;
1H-benz[d]imidazol-2-yl, optionally substituted by phenyl, halo, dihalo, carboxy or methoxycarbonyl;
2- or 3-indolyl, optionally substituted by phenyl, lower alkyl, halo, or acetoxy;
2- or 3-quinolyl, optionally substituted by lower alkyl, halo, or acetoxy; or
phenyl-1,3-thiazol-2-yl, optionally substituted by lower alkyl, halo, or acetoxy, benzo-1,3-thiazol-2-yl or halobenzo-1,3-thiazol-2-yl;
R² is hydrogen;
R³ is alkyl, in particular lower alkyl;
optionally substituted phenyl with one to three substituents, wherein the substituents are selected from the group consisting of halo, cyano, lower alkyl, hydroxy-lower alkyl, phenyl-hydroxy-lower alkyl, optionally halogenated benzyl, methylamino-lower alkyl, dimethylamino-lower alkyl, carbamidoyl-lower alkyl, hydroxy, lower alkoxy, hydroxy-lower alkoxy, phenoxy, benzyloxy, pyridoxy, phenyl, carboxy, phenylcarbonyl, carbamimidoyl, methylsulfonyl, N,N-dimethylsulfamoyl, and N-carbamimidoylsulfamoyl; and
R⁴ is hydrogen or lower alkyl; or
R³ and R⁴ together with the nitrogen atom, to which they are bound, are pyrrolidino, optionally substituted by phenyl, chlorophenyl, benzyl or phenoxy; piperidino, optionally substituted by phenyl, chlorophenyl, benzyl or phenoxy; morpholino; or piperazino, optionally substituted by 4-lower alkyl or 4-benzyl.

4. The compound according to claim 1 of formula (I) wherein
R¹ is optionally substituted biphenylyl, phenoxyphenyl or phenylaminophenyl, in particular halo- or dihalo-biphenylyl, halo- or dihalo-phenoxyphenyl or halo- or dihalo-phenylaminophenyl; 4-(1H-benzimidazol-2-yl)phenyl or 4-(1H-benzimidazol-2-yl)-3-hydroxy-phenyl, wherein benzimidazolyl is optionally substituted at nitrogen by methyl or carboxymethyl and at the benzo residue by carboxy, chloro or dichloro, for example as 5-chloro-, 5-carboxy- or 5,6-dichloro-M-benzimidazol-2-yl;
optionally substituted phenyl-thiophen-2-yl, in particular 3- or 5-phenyl-, 3- or 5-p-fluorophenyl-, 3- or 5-p-chlorophenyl- or 5-(3,4-ethylenedioxyphenyl)-thiophen-2-yl;
optionally substituted benzo[b]thiophen-2-yl, in particular benzo[b]thiophen-2-yl, 3- or 6-chloro-, 3,6-dichloro- or 5,6-ethylenedioxy-3-chloro-benzo[b]thiophen-2-yl;
optionally substituted 1H-benz[d]imidazol-2-yl, in particular 1H-benz[d]imidazol-2-yl, 5- or 6-chloro, carboxy or methoxycarbonyl-M-benz[d]imidazol-2-yl or 5,6-dichloro-M-benz[d]imidazol-2-yl;
optionally substituted 2-indolyl, in particular 2-indolyl, 3-phenyl-, 1-methyl-, 1-methyl-3-phenyl-, 5-chloro-, 5-chloro-3-phenyl-, 6-chloro- or 5,6-dichloro-2-indolyl; or
optionally substituted 2-quinolyl, in particular 2-quinolyl, 6- or 7-chloro- or 6,7-dichloro-2-quinolyl;
R² is hydrogen;
R³ is alkyl, in particular methyl, iso-propyl, iso-butyl or iso-pentyl;
optionally substituted phenyl, in particular 2-, 3- or 4-chlorophenyl, 2- or 4-fluorophenyl, 2-, 3- or 4-bromophenyl, 2-fluoro-4-chlorophenyl, 2,4-difluorophenyl, 3- or 4-cyanophenyl, 3-hydroxymethylphenyl, 3-hydroxymethyl-4-chloro-phenyl, 4-lower alkylphenyl, such as 4-methyl-, 4-iso-propyl, 4-n-butyl-, 4-iso-butyl- or 4-tert-butyl-phenyl, or 4-dimethylaminomethyl-phenyl; and
R⁴ is hydrogen, lower alkyl, in particular methyl, iso-propyl, iso-butyl or iso-pentyl;
or R³ and R⁴ together with the nitrogen atom, to which they are bound, are optionally substituted pyrrolidino, in particular 3-phenyl, 3-p-chlorophenyl-, 3-benzyl- or 3-phenoxy-pyrrolidino, optionally substituted piperidino, in particular 3- or 4-phenoxy-piperidino, 4-phenyl-, 4-p-fluorophenyl-, 4-p-chlorophenyl-, 4-p-hydroxyphenyl-, 4-p-methoxyphenyl- or 4-(2,6-dimethylphenyl)-piperidino, 4-benzyl-, 4-p-fluorobenzyl-, 4-p-chlorobenzyl- or 4-p-hydroxybenzyl-piperidino; morpholino; or optionally substituted piperazino, in particular 4-benzyl- or 4-tert-butyl-piperazino.

5. The compound according to claim 1 of formula (I) wherein
R¹ is p-biphenylyl, 4′-fluoro- or 4′-chloro-(p-biphenylyl), 4-phenoxyphenyl, 4-(m- or p-chlorophenoxy)phenyl, 4-(3,4-dichlorophenoxy)phenyl, 4-(1H-benzimidazol-2-yl)phenyl, (5-chloro- or 5,6-dichloro-M-benzimidazol-2-yl)phenyl; 3- or 5-phenyl- or 5-p-fluorophenyl-thiophen-2-yl; benzo[b]thiophen-2-yl, 3- or 6-chloro-benzo[b]thiophen-2-yl; 2-indolyl, 5-chloro-3-phenyl-2-indanyl; 2-quinolyl, or 6- or 7-chloro- or 6,7-dichloro-2-quinolyl;
R² is hydrogen;
R³ is methyl, iso-propyl, or iso-butyl; 2-, 3- or 4-chlorophenyl, 2- or 4-fluorophenyl, 2-fluoro-4-chlorophenyl, 2,4-difluorophenyl, or 4-methyl-, 4-iso-propyl, 4-n-butyl-, 4-iso-butyl- or 4-tert-butyl-phenyl; and
R⁴ is hydrogen, methyl, iso-propyl, or iso-butyl;
or R³ and R⁴ together with the nitrogen atom, to which they are bound, are 4-phenoxy-piperidino, 4-phenyl-, 4-p-fluorophenyl- or 4-p-chlorophenyl-piperidino, or 4-benzyl-piperidino.

6-7. (canceled)

8. A pharmaceutical composition comprising a compound according to claim 1.

9. A method of treatment and/or prophylaxis of diseases caused by deficiency of synapses, comprising administering to a patient in need thereof a therapeutically effective amount of a compound according to claim 1.

10. A method of treatment and/or prophylaxis of diseases according to claim 9, wherein the diseases caused by deficiency of synapses are skeletal muscle atrophy, schizophrenia, Alzheimer's disease, and cognitive disturbance.
